(19) United States
(12) Patent Application Publication
(10) Pub. No.: US 2010/0318074 A1
(43) Pub. Date: Dec. 16, 2010

(54) OPHTHALMIC ENDOILLUMINATION USING LOW-POWER LASER LIGHT

(76) Inventors: Bruno Dacquay, Irvine, CA (US); Michael J. Yadlowsky, Irvine, CA (US)

Correspondence Address: ALCON, IP LEGAL, TB4-8, 6201 SOUTH FREEWAY, FORT WORTH, TX, 76134, US

(21) Appl. No.: 12/755,479

(22) Filed: Apr. 7, 2010

Related U.S. Application Data
(60) Provisional application No. 61/185,756, filed on Jun. 10, 2009.

(51) Int. Cl. A61F 9/008; A61B 18/20

(52) U.S. Cl. 606/4

(57) ABSTRACT

An ophthalmic surgical system includes a laser light source having a laser treatment mode and an illumination mode. The laser treatment mode has a first power, and the illumination mode has a second power less than the first power. The ophthalmic surgical console also includes focusing optics operable to optically couple the laser light source to a light guide in the illumination mode.

### RELATED APPLICATIONS

This application claims priority to U.S. provisional application Ser. No. 61/185,756, filed on Jun. 10, 2009, the contents which are incorporated herein by reference.

### TECHNICAL FIELD OF THE INVENTION

The present invention relates to an illuminator for use in ophthalmic surgery and more particularly to an ophthalmic endoilluminator to produce a light suitable for illuminating the inside of an eye.

### BACKGROUND OF THE INVENTION

Anatomically, the eye is divided into two distinct parts—the anterior segment and the posterior segment. The anterior segment includes the lens and extends from the outermost layer of the cornea (the corneal endothelium) to the posterior of the lens capsule. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. The vitreous body becomes more fluid with age in a process known as syneresis. Syneresis results in shrinkage of the vitreous body, which can exert pressure or traction on its normal attachment sites. If enough traction is applied, the vitreous body may pull itself from its retinal attachment and create a retinal tear or hole.

Various surgical procedures, called vitreo-retinal procedures, are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions. One typical vitreo-retinal procedure is photocoagulation therapy. In photocoagulation therapy, high-intensity laser light is used to heat proteins in the eye in order repair tears in the retina and to prevent growth of abnormal retinal vasculature that can lead to a detachment. In photocoagulation procedures, a surgeon uses a laser handpiece coupled to a laser source, such as an argon ion laser, to apply the laser light to the target area.

A surgeon performs vitreo-retinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

During such surgical procedures, proper illumination of the inside of the eye is important. Typically, a thin optical fiber is inserted into the eye to provide the illumination. A light source, such as a metal halide lamp, a halogen lamp, a xenon lamp, or a mercury vapor lamp, is often used to produce the light carried by the optical fiber into the eye. The light passes through several optical elements (typically lenses, mirrors, and attenuators) and is emitted to the optical fiber that carries the light into the eye. The quality of this light is dependent on several factors including the types of optical elements selected.

### SUMMARY OF THE INVENTION

In particular embodiments of the present invention, an ophthalmic surgical system includes a laser light source having a laser treatment mode and an illumination mode. The laser treatment mode has a first power, and the illumination mode has a second power less than the first power. The ophthalmic surgical console also includes focusing optics operable to optically couple the laser light source to a light guide in the illumination mode.

In other embodiments of the present invention, a method of illuminating an interior of an eye includes providing a laser light source having a laser treatment mode and an illumination mode. The laser treatment mode has a first power, and the illumination mode has a second power less than the first power. The method further includes optically coupling an endoilluminator handpiece to the laser light source and inserting the endoilluminator handpiece into the eye through a surgical incision. The method then includes illuminating the interior of the eye using laser light from the laser light source in the illumination mode.

Various other aspects of embodiments of the present invention will become apparent from the following detailed description.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 illustrates the anatomy of the eye in which an ophthalmic endoilluminator in accordance with embodiments of the present invention may be placed;

FIG. 2 illustrates an ophthalmic endoilluminator illuminating the interior of the eye in accordance with embodiments of the present invention; and

FIG. 3 is a flowchart illustrating an example method for illuminating an eye using an ophthalmic endoilluminator according to particular embodiments of the present invention.

### DESCRIPTION OF THE INVENTION

Preferred embodiments of the present invention are illustrated in the Figures, like numerals being used to refer to like and corresponding parts of the various drawings.

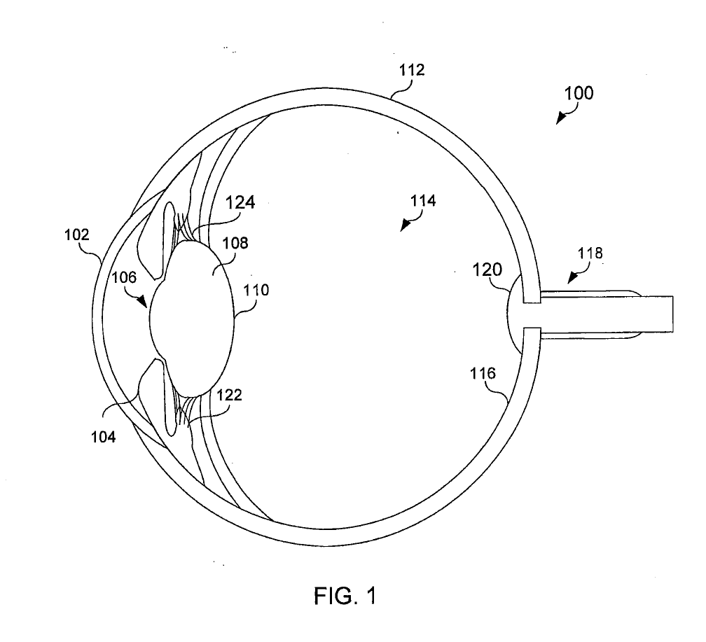

FIG. 1 illustrates the anatomy of the eye into which the improved design for ocular implant provided by the present invention may be placed. Eye 100 includes cornea 102, iris 104, pupil 106, lens 108, lens capsule 110, zonules, ciliary body 120, sclera 112, vitreous gel 114, retina 116, macula, and optic nerve 120. Cornea 102 is a clear, dome-shaped structure on the surface of the eye acts as a window, letting light into the eye. Iris 104 is the colored part of the eye, called the iris, is a muscle surrounding the pupil that relaxes and contracts to control the amount of light entering the eye. Pupil 106 is the round, central opening of the iris. Lens 108 is the structure inside the eye that helps to focus light on the retina. Lens capsule 110 is an elastic bag that envelops the lens, helping to control lens shape when the eye focuses on objects at different distances. Zonules are slender ligaments that attach the lens capsule to the inside of the eye, holding the lens in place. The ciliary body is the muscular area attached to the lens that contracts and relaxes to control the size of the lens for focusing. Sclera 112 is the tough, outermost layer of the eye that maintains the shape of the eye. Vitreous gel 114 is the large, gel-filled section that is located towards the back of the eyeball, and which helps to maintain the curvature of the eye. Retina 116 is a light-sensitive nerve layer in the back of the eye that receives light and converts it into signals to send to the brain. The macula is the area in the retina that contains receptors for seeing fine detail. Optic nerve 118 connects and transmits signals from the eye to the brain.

Ciliary body 122 lies just behind the iris 104. Attached to the ciliary body 122 are tiny fiber “guide wires” called zonules 124. Lens 108 is suspended inside the eye by the zonular fibers 124. Nourishment for the ciliary body 122 comes from blood vessels which also supply the iris 104. One function of ciliary body 122 is to control accommodation by changing the shape of the lens 108. When the ciliary body 122 contracts, the zonules 124 relax. This allows the lens 108 to thicken, increasing the eye's ability to focus up close. When looking at a distant object, ciliary body 122 relaxes, causing the zonules 124 to contract. The lens 108 then becomes thinner, adjusting the eye's focus for distance vision.

Ordinarily, the retina 116 is protected from ultraviolet light by the eye's natural lens 108, which filters the light that enters the eye. But light from an optical endoilluminator enters the eye without this lens filtration (i.e., aphakically), and if this light includes sufficiently intense components near the ultraviolet range or infrared range of the electromagnetic spectrum, it can damage ophthalmic tissue. Providing light of the proper range of visible light wavelengths for illumination while filtering out harmful short and long wavelengths can greatly reduce the risk of damage to the retina through aphakic hazards, including blue light photochemical retinal damage, infrared heating damage, and similar light toxicity hazards. Typically, a light in the range of about 430 to 700 nanometers is preferable for reducing the risks of these hazards.

In order to achieve sufficient light intensity, however, previous ophthalmic endoilluminators have been based on broad-spectrum light sources. For example, many endoillumination light sources use halogen tungsten lamps or high pressure arc lamps (metal-halides, Xe). The advantages of arc lamps are small emitting area (<1 mm), color temperature close to daylight, and longer life than in halogen lamps—400 hours vs. 50 hours. The disadvantage of arc lamps is high cost, decline in power, complexity of the systems and the need to exchange lamps several times over the life of the system. LED based illuminators may provide considerably lower cost and complexity, and characteristic life times of 50,000 to 100,000 hours that would allow operating ophthalmic fiber illuminator for entire life of the instrument with very little drop in output and without a need of exchanging LEDs. A typical white LED may include ultra violet (UV)/violet/blue LED exciting a white phosphor cap to produce enough white light for the endoilluminator.

Unlike conventional illuminators, various embodiments of the present invention provide illumination using low-power laser light. This provides sufficient illumination intensity in the visible light spectrum while avoiding components of the electromagnetic spectrum that can be harmful to ocular tissue. Advantageously, the wavelength of light used in the low-power laser illuminator can be selected to improve contrast in the visualized area. Thus, for example, a laser source used in certain photocoagulators, such as the PUREPOINT® photocoagulator produced by Alcon Laboratories, Inc., can produce green laser light having a wavelength of about 532 nm (the term “about” used herein to mean consistent generation of laser light within +/−5 nm of a nominal wavelength). As compared to previous endoilluminators, the light and dark areas resulting from absorption of light of this wavelength can improve the visual contrast between retinal vasculature and other optical tissue.

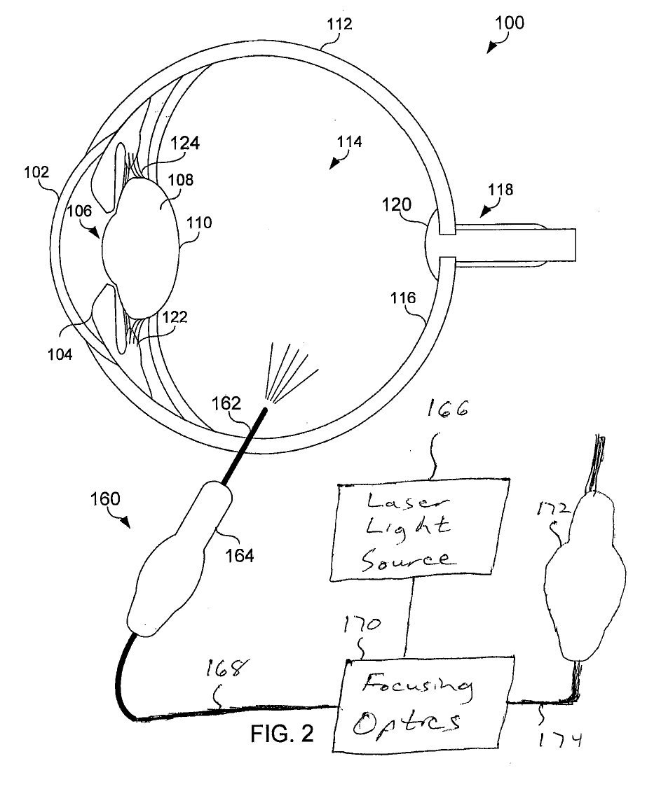

FIG. 2 is a cross sectional view of an ophthalmic endoilluminator 160, which may be an endoilluminator according to any of the various embodiments of the present invention, located in an eye. FIG. 2 depicts handpiece 164 with handpiece 162 in use. Handpiece 162 is inserted into eye 100 through an incision in the pars plana region. Handpiece 162 illuminates the inside or vitreous region 114 of eye 100. In this configuration, handpiece 162 can be used to illuminate the inside or vitreous region 114 during vitreo-retinal surgery. Handpiece 162 is connected to a laser light source 166 by a light guide 168, which is typically an optical fiber. Focusing optics 170 couple the laser beam emitted from laser light source 166 to light guide 168. The focusing optics 170 may be located either internal or external to the laser light source 166 or an associated ophthalmic surgical console. Light guide 168 may include any conduit suitable for carrying light of a wavelength produced by laser light source 166, having any desired core, cladding, dopants, refractive index, thermal properties, mechanical properties, or other characteristics known in the art. Glass or plastic optical fibers used in ophthalmic applications typically range from 50-300 μm in diameter for fibers used to deliver treatment radiation and from 400-750 μm for fibers used to deliver illumination.

Laser light source 166 may be any suitable device for producing coherent laser light of a wavelength in the visible spectrum of sufficient intensity to allow visualization of ocular tissue. In a particular embodiment, laser light source 166 produces green laser light having a wavelength around 532 nm. Laser light source 166 may also be coupled to a laser treatment handpiece 172, which may also include a respective light guide 174 similar to the one described for endoilluminator handpiece 162 but suitable for carrying laser light used for producing photochemical changes in ocular tissue. Focusing optics 170 may also include separate and/or components for coupling laser light source 166 to laser treatment handpiece 172. In particular embodiments, endoilluminator handpiece 162 and laser treatment handpiece 172 could be integrated into a single combined handpiece.

In operation, the laser light source 166 has two different operational modes. The first mode is a laser treatment mode having a power density for the laser beam impinging on the ocular tissue sufficient to produce photochemical changes, such as by thermal effects produced by absorption of the laser light, within a relatively small area of the ocular tissue targeted by the beam spot. In particular embodiments, such photochemical changes can be used to repair tears or detachments in retinal tissue or to inhibit growth of abnormal vasculature in the retina. In particular embodiments, the laser treatment mode may be a photocoagulation mode that produces coagulation of retinal tissue by thermal changes in the proteins of the optical tissue. The second mode is an illumination mode. In the illumination mode, laser light is used to illuminate a surgical field around a target site for a surgical operation. The illumination mode uses a lower power so that the properties of the retinal tissue are unchanged. In most applications, the spot size will also be substantially larger than the spot size for the laser treatment mode in order to provide a view of the area surrounding the target site for the surgical operation, but in narrow-angle illumination applications, the spot size might be comparable.

In one example, the laser light source 166 is also used for photocoagulation. In a typical photocoagulation application, the laser power used to produce thermal changes in the ocular tissue is at least 100 mW for a spot size on the order of 1 mm at the retina, with the laser beam being emitted at an estimated working distance of 5 mm and being transmitted in a balanced saline solution medium. In such an application, the laser light source 166 may, for example, be used to generate a spot size of 50 μm or less coupled an optical fiber with a numerical aperture to produce a spot size of 1 mm at the retina.

Unlike the intensity required for photocoagulation, there is no clear minimum intensity required for adequate illumination, since what is required may vary from surgeon to surgeon. Previous commercial illuminators used in ophthalmic surgery have produced a luminous flux at the surgical field of up to 12 lumens for ordinary illumination and of 15 lumens or more for wide-angle illumination, but flux levels of even 10% of the maximum could be adequate, particularly when the endoilluminator handpiece is advantageously configured so the that effective irradiance of the target surgical site as a function of the luminous flux produced by the endoilluminator is comparatively high as compared to a point source.

Given general requirements such as those outlined above, the power level for the illumination mode of the laser light source 166 can be selected. Lasers often have a relatively high conversion efficiency for the characteristic wavelength, so a high level of flux can ordinarily be generated with a relatively low power. Thus, for a readily achievable conversion efficiency of around 600 lumens/W, the power required to produce the same maximum flux would be only about 20-25 mW. Typical ophthalmic laser light sources for photocoagulators operate in the range of 100-600 mW. But there are some existing laser light sources, such as the PUREPOINT® laser light source manufactured by Alcon Laboratories, Inc., with an operating range from about 30 mW to 2 W, with the lower end being near the peak power of existing illuminators, that could be made to function in illumination mode without a large degree of modification. For narrow angle applications, the power level could in principle be even lower, and in general, a power level between 10 nW and 50 mW would be a preferred range covering suitable power levels for many applications.

In terms of reducing aphakic hazard to ocular tissue, the risk of eye damage can be comparatively assessed with conventional endoilluminators using white light illumination. A conventional white-light endoilluminator is considered aphakically safe with flux levels in the range of 12-15 lumens, as noted above. Retinal tissue damage has not been noted for such instruments even when used in surgery lasting longer than an hour. With a laser having a narrow emission profile around a single wavelength, the components of the spectrum in the aphakically hazardous range are significantly less intense. For example, if laser light of 532 nm were compared to a Xe bulb illuminator, the total irradiance on the retina of aphakically hazardous electromagnetic radiation would be reduced by a factor of almost 12. Thus, for the same degree of illumination, the risk of damage to ocular tissue should be even less than for conventional endoilluminators.

One difficulty unique to laser light illuminators is the possibility of thermal damage to light guide 168. Endoilluminators typically use plastic optical fibers that are flexible to allow easy placement of the endoilluminator within the eye. Light is coupled into the plastic illuminator with a relatively high numerical aperture (NA) of the beam, typically around 0.5, to produce a sufficiently large spot size at the surgical field. But laser beams used in applications like photocoagulation are often emitted with a spot size so small that coupling to a fiber with such a high numerical aperture would produce an extremely intense irradiance at the beam waist, even at relatively low laser power. The absorption of this intense irradiance by the plastic optical fiber can heat the plastic above its melting temperature, causing fiber breakdown.

Consequently, rather than using the conventional optics for coupling white light sources to endoilluminator fibers, the focusing optics 170 of laser endoilluminator 160 should be configured to prevent spots of intense irradiance from forming on a plastic endoilluminator fiber. To prevent such intense spots on the fiber, it is advantageous to broaden the size of the incident beam to fill the fiber aperture as nearly as possible while maintaining the desired NA. Thus, for example, a cylindrical quartz rod can be placed with a proximal end at the laser beam focus and a distal end butted against a proximal end of the light guide 168, which will diffuse the beam to a considerable larger spot size without significantly reducing the total intensity of light being delivered to the light guide 168. In another example, a scattering plate could be used.

In some cases, it may also be desirable to have a relatively narrow field of illumination, which can be useful for illuminating certain structures. In such cases, the laser light source 166 might be coupled to a fiber used for treatment with a lower numerical aperture while the laser light source 166 is the illumination mode, so as to produce a relatively small illumination spot at a much lower intensity than a treatment beam. Such embodiments may allow the laser light source 166 to be switched between treatment and illumination while a laser treatment handpiece 172 is within the eye during surgery, thus providing illumination and treatment with a single handpiece 172 without the need for separate illumination and treatment fibers in the handpiece 172.

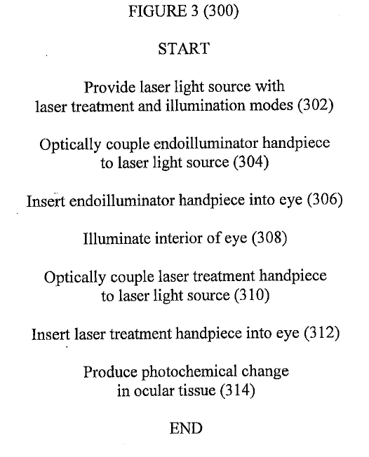

FIG. 3 is a flowchart 300 illustrating an example method for illuminating an eye with an optical endoilluminator according to particular embodiments of the present invention. At step 302, a laser light source 166 is provided having a laser treatment mode and an illumination mode as described in conjunction with the various embodiments above. At step 304, an endoilluminator handpiece 162 is optically coupled to the laser light source 166. At step 306, the handpiece 162 is inserted with an eye through a surgical incision. At step 308, an interior of the eye is illuminated using the handpiece.

At step 310, a laser treatment handpiece 172 is optically coupled to the laser light source 166, and the laser treatment handpiece 172 is inserted into the eye through an incision at step 312. At step 314, a photochemical change in tissue of the eye is produced using laser light from the laser light source 166. Although a particular method has been described in detail, it should be appreciated that various steps could be rearranged and/or omitted in a manner consistent with the various embodiments of an ophthalmic surgical system described above, and additional steps might be added. Accordingly, any suitable method of use for such ophthalmic surgical systems is contemplated within the scope of this disclosure.

The present invention is illustrated herein by example, and various modifications may be made by a person of ordinary skill in the art. For example, the low power modes of the laser light source may be achieved by coupling attenuator accessories to the laser light source in order to produce a certain output power level to the handpiece. Although the present invention is described in detail, it should be understood that various changes, substitutions and alterations can be made hereto without departing from the scope of the invention as claimed.

What is claimed is:

1. An ophthalmic surgical system, comprising:
a laser light source having a laser treatment mode and an illumination mode, the laser treatment mode having a first power and the illumination mode having a second power less than the first power; and
focusing optics operable to optically couple the laser light source to a light guide in the illumination mode.

2. The system of claim 1, wherein the first power is at least 100 mW, and the second power is in a range from 10 nW to 50 mW.

3. The system of claim 1, wherein the laser light source produces laser light with a wavelength in a range from 430 to 700 nm.

4. The system of claim 1, wherein the laser light source produces laser light with a wavelength of 532 nm.

5. The system of claim 1, wherein the laser light source produces laser light having a first spot size, and the focusing optics broaden the laser light in the illumination mode to a second spot size larger than the first spot size.

6. The system of claim 1, wherein the focusing optics comprise a cylindrical quartz rod.

7. The system of claim 1, further comprising a laser treatment handpiece coupled to the laser light source.

8. The system of claim 7, wherein the laser treatment handpiece is configured to produce photocoagulation of retinal tissue using laser light produced by the laser light source in the laser treatment mode.

9. The system of claim 7, wherein the laser treatment handpiece is configured to produce photochemical changes in ocular tissue within a circular area less than 1 mm in diameter from a distance of 5 mm.

10. The system of claim 1, further comprising a light guide coupled to the laser light source and an endoilluminator handpiece coupled to the light guide.

11. The system of claim 10, wherein the endoilluminator handpiece is configured to illuminate an area at least 12 mm in diameter within an eye.

12. A method of illuminating an interior of an eye, comprising:
providing a laser light source having a laser treatment mode and an illumination mode, the laser treatment mode having a first power and the illumination mode having a second power less than the first power;
optically coupling an endoilluminator handpiece to the laser light source;
inserting the endoilluminator handpiece into the eye through a surgical incision; and
illuminating the interior of the eye using laser light from the laser light source in the illumination mode.

13. The method of claim 12, further comprising:
optically coupling a laser treatment handpiece to the laser light source; and
producing a photochemical change in tissue of the eye using laser light from the laser light source in the laser treatment mode.

14. The method of claim 13, wherein the photochemical change is photocoagulation.

15. The method of claim 12, wherein the first power is at least 100 mW, and the second power is in a range from 10 nW to 50 mW.

16. The method of claim 12, wherein the laser light source produces laser light with a wavelength in a range from 430 to 700 nm.

17. The method of claim 16, wherein the laser light source produces laser light with a wavelength of 532 nm.

18. The method of claim 12, wherein the step of illuminating comprises illuminating an area at least 12 mm in diameter.

19. An ophthalmic surgical system, comprising:
a laser light source having a laser treatment mode and an illumination mode, the laser treatment mode having a first power at least 100 mW and the illumination mode having a second power between 10 nW and 50 mW, the laser light source operable to produce laser light having a wavelength about 532 nm;
an endoilluminator handpiece; and
focusing optics optically coupling the laser light source to a light guide in the endoilluminator handpiece.

20. The system of claim 19, further comprising a laser treatment handpiece optically coupled to the laser light source, the laser treatment handpiece configured to perform photocoagulation of ocular tissue using laser light from the laser light source in the laser treatment mode.
